(19) United States
(12) Patent Application Publication
(10) Pub. No.: US 2024/0423551 A1
(43) Pub. Date: Dec. 26, 2024

(54) METHOD AND SYSTEM FOR MONITORING BODY MOVEMENTS

(71) Applicant: Soter Analytics Pty Ltd, Como (AU)

(72) Inventors: Matthew HART, Como (AU); Alexey PAVLENKO, Como (AU); Anastasia VASINA, Como (AU)

(73) Assignee: Soter Analytics Pty Ltd, Como (AU)

(21) Appl. No.: 18/686,483

(22) PCT Filed: Aug. 25, 2022
(86) PCT No.: PCT/AU2022/051039

§ 371 Date: Feb. 26, 2024

(30) Foreign Application Priority Data
Aug. 25, 2021 (AU) 2021902777

(51) Int. Cl. A61B 5/00; A61B 5/11

(52) U.S. Cl. CPC A61B 5/7275; A61B 5/1114; A61B 5/1116; A61B 5/1121; A61B 5/1126; A61B 5/6814; A61B 5/6823; A61B 5/6824; A61B 2503/20; A61B 2560/0257; A61B 2562/0219

(57) ABSTRACT

A system for monitoring body movement, comprising an inertial motion sensing unit arranged to measure movement of a body part, the sensing unit arranged to iteratively collect data representing movement of the body part over time; and a processor for receiving and analysing the data to determine risk of injury of the body part and/or to track the movement of the body part.

### FIELD OF THE INVENTION

The present invention relates to a method and system for monitoring body movements.

### BACKGROUND

Physically demanding jobs expose workers to prolonged manual handling activities, such as lifting, holding or moving loads. These manual handling activities can lead to negative health effects and manual handling injuries, such as back pain. The effect of manual handling injuries caused through instantaneous injuries or by gradual wear and tear, has a significant social, human and organisational cost for both workers and workplace organisations.

Workplace organisations have duties to protect workers from the risk of manual handling injuries. These duties include identifying tasks that involve hazardous manual handling, assessing the risk of these tasks, and reducing or eliminating the risks through risk control mechanisms.

Risk control mechanisms may be in the form of training programs for educating workers about proper manual handling practices, training manuals for referencing, and mechanical aids for reducing physical efforts. However, the implementation of risk control mechanisms does not ensure the elimination of manual handling injuries. Once implementation has occurred, continuous monitoring of worker habits and adoption of the risk control mechanisms is necessary.

Safety officers are typically employed to monitor worker habits and their adoption of the risk control mechanisms. These safety officers are required to warn workers when they are exercising poor manual handling practices, provide re-education when workers revert to poor manual handling practices, and report incidents to the organisation. However, safety officers may not be present or made aware of incidents where workers exhibit poor manual handling practices and/or incur a manual handling injury.

The present invention seeks to overcome, or at least substantially ameliorate, the disadvantages and shortcomings of the background art.

Any references to documents that are made in this specification are not intended to be an admission that the information contained in those documents form part of the common general knowledge known to a person skilled in the field of the invention, unless explicitly stated as such.

In this specification the terms “comprising” or “comprises” are used inclusively and not exclusively or exhaustively.

### SUMMARY OF THE INVENTION

According to a first aspect of the invention, there is provided a system for monitoring body movement, comprising:
an inertial motion sensing unit arranged to measure movement of a body part, the sensing unit arranged to iteratively collect data representing movement of the body part over time; a processor for receiving and analysing the data to determine risk of injury of the body part and/or to track the movement of the body part.

In an embodiment, the processor configured to:
determine an orientation of the body part;
maintain a record of a state of movement of the body part;
determine whether the body part has a changed state of movement and to update the maintained record of the state of movement when the body part has the changed state of movement;
classify the movement of the body part according to the orientation of the body part, the current state of movement of the body part and the data during the current state of movement of the body part.

In an embodiment, the processor is configured to determine a type of movement the body part is making from data collected during tracking of the movement of the body part. In an embodiment, the processor is configured to learn one or more characteristics of movement of the body part for each type of movement from the data collected during tracking of the movement of the body part. Preferably the characteristics determine a “correct” type of implementation and an “incorrect” type of implementation of the determined type of movement the body part.

In an embodiment, the processor is configured to determine whether the movement the body part is undertaking is the “correct” type of implementation of the determined type of movement and determine whether the movement the body part is undertaking is the “incorrect” type of implementation of the determined type of movement the body part is making according to the data collected during tracking of the movement of the body part during or following each movement.

In an embodiment, the configuration to determine the orientation of the body part comprises applying one or more filters to the measured movement of the body part. Preferably, the one or more filters comprise a Madgwick filter.

In an embodiment, the configuration to determine whether the body part has the changed state of movement comprises determining whether the body part has started moving, or has finished moving, and the direction of movement relative to the determined orientation of the body part when the body part is moving.

In an embodiment, the analysis comprises determining an angle of an arc traced by movement of an extent of the body part (angle of movement). For example, the extent may be an axial length of the upper arm and the arc may be traced by the elbow when the pivot point of the movement is the shoulder socket. Alternatively, the extent may be the length of the spine and the arc may be traced by the neck when the pivot point is at the waist.

In an embodiment, the analysis comprises determining an angle of an arc traced by movement of the body part when it changes orientation (angle of orientation). For example, the arm may be in a horizontal orientation and changes to a vertical orientation (such that the angle is 90 degrees). The orientation may be in one or two dimensions. Alternatively, the back may be vertical (shoulder blades facing horizontal) and the change in orientation may be to horizontal, in a straight bend down. Additionally, the orientation of the shoulder blades may be at an angle to vertical when there is also twisting.

In an embodiment, the analysis comprises determining a neutral starting position of a movement of the body part. In an embodiment, the determination of the angle(s) is taken from the neutral starting position, such as standing straight or sitting straight, with movement tracked in 3D space from the neutral starting position.

In an embodiment, the processor is configured to detect false-positive movements of the body using the collected data.

In an embodiment, the analysing comprises determining whether a determined state change is a false positive. In an embodiment, determining of the false positive comprises analysing one or more of: a duration of a detected movement, a change in the angle of movement, an orientation of the body part, a rate of change of the angle of movement, a rate of change of the angle of orientation. In an embodiment, determining the false positive comprises determining whether there is an atmospheric pressure change over the course of the movement of the body part. In an embodiment, the pressure change is measured to determine whether the body part altered its altitude as determined by the change in atmospheric pressure.

In an embodiment, the classification comprises determining a tilting or movement technique performed by the body part. In an embodiment, determining the technique comprises analysing the change in the angle of movement, and/or the orientation of the body part and/or determining whether there is an atmospheric pressure change.

In an embodiment, the classification comprises determining whether there is a transition from standing to sitting or a transition from sitting to standing. In an embodiment, determining whether there is a transition to/from sitting comprises providing the inertial motion data and/or data derived therefrom to a trained classifier and receiving the output of the classifier as the determination or a precursor thereof. In an alternative embodiment, determining whether there is a transition to/from sitting comprises applying a set of one or more condition checks based on the inertial motion data and/or data derived therefrom.

In an embodiment, the system further comprises a transmitter for transmitting the collected data.

In an embodiment, the sensing unit further comprises an atmospheric pressure sensor for measuring atmospheric pressure external to a housing of the sensing unit. In an embodiment, the processor is configured to detect false-positive movements of the body using measurements of atmospheric pressure external to the housing at different times in the course of a movement of the body part.

In an embodiment, the processor is configured to determine from the data which body part the housing is attached to. In an embodiment, the determination determines that the housing is located on one of the following: the user's trunk and the user's arm.

In an embodiment, the processor is configured to compare the collected data to expected movements in a model of body part movements to determine from the collected data which body part the body is attached to. In an embodiment, the model is created based on the collected data for a plurality of movements of the same body part being determined to perform the same type of movement of the body part.

In an embodiment, the processor is configured to analyse the data to determine when a lift is occurring. In an embodiment, the processor is configured to analyse the data to determine a lifting technique used when a lift is occurring. In an embodiment, the processor is configured to analyse the data to determine arm movement when a lift is occurring.

In an embodiment, the processor is configured to analyse the data to determine when a user is sitting. In an embodiment the processor is configured to analyse the data to determine arm movement when the user is sitting.

In an embodiment, the processor is configured to process data from the accelerometer and the gyroscope with a Madgwick filter to determine an orientation of the body part.

In an embodiment, the processor is configured to determine a start point of the

movement of the body part. In an embodiment, the processor is configured to determine an end point of the movement of the body part.

In an embodiment, the processor is configured to determine an intensity of force in a movement of the body part.

In an embodiment, the processor is configured to determine a state of the body part. In an embodiment, the state is one or more of: moving, lifting, bending, twisting, twisting while bending and stationary.

In an embodiment, the processor is configured to determine that during movement of the body part, the movement is jerky.

In an embodiment, the processor is configured to determine repetition of the same body part movement over a period of time.

In an embodiment, the processor is configured to determine staying in a static position during a movement of the body part.

In an embodiment, the processor is configured to determine a RULA assessment. In an embodiment, the processor is configured to determine a REBA assessment.

In an embodiment, the processor is configured to determine a NIOSH lifting assessment. In an embodiment, the processor is configured to determine a WISHA lifting assessment.

In an embodiment, the processor is configured to determine a number of movements of the body part over a time period (eg. an hour).

In an embodiment, the processor is configured to determine a work pattern from a plurality of movements. In an embodiment, determining a work pattern comprises determining the number and amount of intensities of movement. In an embodiment determining a work pattern comprises determining an average amount of time spent moving in each movement and an average of the amount of time spent recovering from each movement.

In an embodiment, the processor is configured to determine a shoulder limb position angle, velocity of movement, jerkiness of movement, hazards per hour, frequency of arm elevation, proportion of time arm elevated, intensity of force of movement over time, amount of time spent moving, amount of time spent recovering.

In an embodiment, the processor is configured to determine a back position, velocity of movement, jerkiness of movement, twisting while bending angle, bending angle, hazards per hour, frequency of bending, twisting, static posture and intensity movement, proportion of time bending twisting, intensity of force of movement twisting while bending.

In an embodiment, the processor is configured to classify movements as one or more of the following: arm elevated more than 90 degrees; arm elevated more than 90 degrees for a period in excess of 30 seconds; arm elevated more than 90 degrees more than 2 times a minute; arm elevated for than 90 degrees for more than 20% of working time; hazardous pulling and pushing.

In an embodiment, the processor is configured to classify movements as one or more of the following: twisting of back more than 30 degrees while bending more than 50 degrees; bending more than 90 degrees; bending more than 60 degrees for at least 20 seconds; at least 2 hazardous movement in 2 minutes; jerky movement.

In an embodiment, the processor is configured to determine a risk of continued performance of the movement of the body part. In an embodiment, the risk is a determination of risk of back injury. In an embodiment, the risk is a determination of risk of shoulder injury. In an embodiment, the risk is a determination of risk of back and shoulder injury. In an embodiment the determination of risk is based on one or more of the previously described determinations.

According to a second aspect of the invention, there is provided a system for monitoring body movement, comprising:
an inertial motion unit sensor arranged to measure movement of a body part, the sensor arranged to iteratively collect data representing movement of the body part over time;
a processor for receiving and analysing the data, the processor configured to:
determine an orientation of the body part;
maintain a record of a state of movement of the body part;
determine whether the body part has a changed state of movement and to update the maintained record of the state of movement when the body part has the changed state of movement;
classify the movement of the body part according to the orientation of the body part, the current state of movement of the body part and the data during the current state of movement of the body part.

According to a third aspect of the invention, there is provided a method of monitoring body movement, comprising:
measuring movement of a body part by iteratively collect data representing movement of the body part over time using a sensor unit;
analysing the data to determine risk of injury of the body part or to track the movement of the body part.

In an embodiment, the analysing step comprising:
determining an orientation of the body part;
maintaining a record of a state of movement of the body part;
determining whether the body part has a changed state of movement and to update the maintained record of the state of movement when the body part has the changed state of movement;
classifying the movement of the body part according to the orientation of the body part, the current state of movement of the body part and the data during the current state of movement of the body part.

In an embodiment, the method comprises determining a type of movement the body part is making from tracking of the movement of the body part. In an embodiment, the method comprises leaning one or more characteristics of movement of the body part for each type of movement from the tracking of the movement of the body part. Preferably the characteristics determine a “correct” type of implementation and an “incorrect” type of implementation of the determined type of movement the body part.

In an embodiment, the method comprises determining when the movement of the body part is undertaking the “correct” type of implementation of the determined type of movement and when the movement of the body part is undertaking the “incorrect” type of implementation of the determined type of movement the body part is making according to the tracking of the movement of the body part during each movement.

In an embodiment, the method comprises determining the orientation of the body part comprises applying one or more filters to the measured movement of the body part. Preferably, the one or more filters comprise a Madgwick filter.

In an embodiment, the method comprises determining whether the body part has the changed state of movement comprises determining whether the body part has started moving, or has finished moving, and the direction of movement relative to the determined orientation of the body part when the body part is moving.

In an embodiment, the method comprises determining an angle of an arc traced by movement of an extent of the body part (angle of movement).

In an embodiment, the method comprises determining an angle of an arc traced by movement of the body part when it changes orientation (angle of orientation).

In an embodiment, the method comprises determining a neutral starting position of a movement of the body part. In an embodiment, determining the angle is taken from the neutral starting position, with movement tracked in 3D space from the neutral starting position.

In an embodiment, the method comprises detecting false-positive movements of the body using the collected data.

In an embodiment, the method comprises determining whether a determined state change is a false positive. In an embodiment, determining of the false positive comprises analysing one or more of: a duration of a detected movement, a change in the angle of movement, an orientation of the body part, a rate of change of the angle of movement, a rate of change of the angle of orientation. In an embodiment, determining the false positive comprises determining whether there is an atmospheric pressure change over the course of the movement of the body part. In an embodiment, the pressure change is measured to determine whether the body part altered its altitude as determined by the change in atmospheric pressure.

In an embodiment, the method comprises determining a tilting or movement technique performed by the body part. In an embodiment, determining the technique comprises analysing the change in the angle of movement, and/or the orientation of the body part and/or determining whether there is an atmospheric pressure change.

In an embodiment, the method comprises determining whether there is a transition from standing to sitting or a transition from sitting to standing. In an embodiment, determining whether there is a transition to/from sitting comprises providing the inertial motion data and/or data derived therefrom to a trained classifier and receiving the output of the classifier as the determination or a precursor thereof. In an alternative embodiment, determining whether there is a transition to/from sitting comprises applying a set of one or more condition checks based on the inertial motion data and/or data derived therefrom.

In an embodiment, the method comprises transmitting the collected data.

In an embodiment, the method comprises measuring atmospheric pressure. In an embodiment, the method comprises detecting false-positive movements of the body using measurements of atmospheric pressure at different times in the course of a movement of the body part.

In an embodiment, the method comprises determining from the data which body part a housing of a sensor unit is attached to. In an embodiment, the determination determines that the housing is located on one of the following: the user's trunk and the user's arm.

In an embodiment, the method comprises comparing the data to expected movements in a model of body part movements to determine from the data which body part the body is attached to. In an embodiment, the method comprises creating the model based on the collected data for a plurality of movements of the same body part being determined to perform the same type of movement of the body part.

In an embodiment, the method comprises analysing the data to determine when a lift is occurring. In an embodiment, the method comprises analysing the data to determine a lifting technique used when a lift is occurring. In an embodiment, the method comprises analysing the data to determine arm movement when a lift is occurring.

In an embodiment, the method comprises analysing the data to determine when a user is sitting. In an embodiment the method comprises analysing the data to determine arm movement when the user is sitting.

In an embodiment, the method comprises processing data from an accelerometer and a gyroscope with a Madgwick filter to determine an orientation of the body part.

In an embodiment, the method comprises determining a start point of the movement of the body part. In an embodiment, the method comprises determining an end point of the movement of the body part.

In an embodiment, the method comprises determining an intensity of force in a movement of the body part.

In an embodiment, the method comprises determining a state of the body part. In an embodiment, the state is one or more of: moving, lifting, bending, twisting, twisting while bending and stationary.

In an embodiment, the method comprises determining whether during movement of the body part, the movement is jerky.

In an embodiment, the method comprises determining whether there is repetition of the same movement of the body part over a period of time.

In an embodiment, the method comprises determining whether the body part stays in a static position during a movement of the body part.

In an embodiment, the method comprises determining a RULA assessment. In an embodiment, the method comprises determining a REBA assessment. In an embodiment, the method comprises determining a NIOSH lifting assessment. In an embodiment, the method comprises determining a WISHA lifting assessment.

In an embodiment, the method comprises determining a number of movements of the body part over a time period (eg. an hour).

In an embodiment, the method comprises determining a work pattern from a plurality of movements. In an embodiment, determining a work pattern comprises determining the number and amount of intensities of movement. In an embodiment determining a work pattern comprises determining an average amount of time spent moving in each movement and an average of the amount of time spent recovering from each movement.

In an embodiment, the method comprises determining a shoulder limb position angle, velocity of movement, jerkiness of movement, hazards per hour, frequency of arm elevation, proportion of time arm elevated, intensity of force of movement over time, amount of time spent moving, amount of time spent recovering.

In an embodiment, the method comprises determining a back position, velocity of movement, jerkiness of movement, twisting while bending angle, bending angle, hazards per hour, frequency of bending, twisting, static posture and intensity movement, proportion of time bending twisting, intensity of force of movement twisting while bending.

In an embodiment, the method comprises classifying movements as one or more of the following: arm elevated more than 90 degrees; arm elevated more than 90 degrees for a period in excess of 30 seconds; arm elevated more than 90 degrees more than 2 times a minute; arm elevated for than 90 degrees for more than 20% of working time; hazardous pulling and pushing.

In an embodiment, the method comprises classifying movements as one or more of the following: twisting of back more than 30 degrees while bending more than 50 degrees; bending more than 90 degrees; bending more than 60 degrees for at least 20 seconds; at least 2 hazardous movement in 2 minutes; jerky movement.

In an embodiment, the method comprises determining a risk of continued performance of the movement of the body part. In an embodiment, the risk is a determination of risk of back injury. In an embodiment, the risk is a determination of risk of shoulder injury.

In an embodiment, the risk is a determination of risk of back and shoulder injury.

According to a fourth aspect of the invention, there is provided a method of monitoring body movement, comprising:
measuring movement of a body part by iteratively collect data representing movement of the body part over time using a sensor unit;
determining an orientation of the body part;
maintaining a record of a state of movement of the body part;
determining whether the body part has a changed state of movement and updating the maintained record of the state of movement when the body part has the changed state of movement;
classifying the movement of the body part according to the orientation of the body part, the current state of movement of the body part and the data during the current state of movement of the body part.

According to another aspect there is a sensor device for monitoring body part movements, comprising:
a housing having an attachment device for attaching the housing to a body part;
an accelerometer mounted in the housing;
a gyroscope mounted in the housing;
a processor for collecting data of measurements from the accelerometer, and the gyroscope.

In an embodiment the sensor device further comprises a transmitter for transmitting the collected data.

In an embodiment the sensor device further comprises an atmospheric pressure sensor for measuring atmospheric pressure external to the housing.

In an embodiment the processor is configured to detect false-positive movements of the body using the collected data. In an embodiment the processor is configured to detect false-positive movements of the body using measurements of atmospheric pressure external to the housing at different times in the course of a movement of the body part.

In an embodiment the housing is configured to attach to the user's arm. In an embodiment the housing is configured to attach to the user's torso, preferably their back in the region of between their shoulder blades.

In an embodiment the processor is configured according to one or more of the configurations of the previous aspects.

According to another aspect there is a sensor device for monitoring body part movements, comprising:
a housing having an attachment device for attaching the housing to a body part;
an accelerometer mounted in the housing;
a gyroscope mounted in the housing;
an atmospheric pressure sensor mounted in the housing and arranged to measure external atmospheric pressure;
a processor for collecting data of measurements from the accelerometer, the gyroscope and the atmospheric pressure sensor.

According to another aspect of the invention, there is provided a method of monitoring body movement, comprising:
attaching an inertial motion sensing unit to a body part
iteratively collecting data representing movement of the body part over time from the sensing unit;
determining an orientation of the body part from the collected data;
maintaining a record of a state of movement of the body part;
determining whether the body part has a changed state of movement;
updating the maintained record of the state of movement when the body part has the changed state of movement;
classifying the movement of the body part according to the orientation of the body part, the current state of movement of the body part and the data during the current state of movement of the body part.

According to a further aspect there is provided a method of monitoring body part movements, comprising:
attaching a housing of a monitoring device to a body part;
collecting data from an accelerometer mounted in the housing;
collecting data from a gyroscope mounted in the housing;
collecting data from an atmospheric pressure sensor mounted in the housing and arranged to measure external atmospheric pressure;
processing the collected data.

According to a further aspect of the invention, there is provided a computer program product, comprising a set of instructions for controlling a processor to: operate the processor as defined above.

According to an eleventh aspect of the invention, there is provided a computer program product, comprising a set of instructions for controlling a processor to: perform the method as defined above.

### BRIEF DESCRIPTION OF THE DRAWINGS

Embodiments of the present invention are described in the following detailed description by example only, with reference to the following drawings:

FIG. 1 illustrates an example of a system embodying the present invention;

FIG. 1A is a schematic view of an alternative clip-on type of monitoring device of the system of FIG. 1;

FIG. 1B shown a further alternative clip-on type of monitoring device of the system of FIG. 1;

FIG. 2 is a block diagram of components of a monitoring device of the system of FIG. 1;

FIG. 3 is a block diagram of components of a remote computing resource of the system of FIG. 1;

FIG. 4 is a block diagram of components of a personal computing resource of the system of FIG. 1;

FIG. 5 is a block diagram of functional modules of the monitoring device of FIG. 2;

FIG. 6 is a block diagram of functional modules of the remote computing resource of FIG. 3;

FIG. 7 is a block diagram of functional modules of the personal computing resource of FIG. 4;

FIG. 8 is a flowchart illustrating a method of monitoring core body movement;

FIG. 9A is a display of spine hazards generated by an example of an embodiment of the present invention;

FIG. 9B is a display of shoulder hazards generated by an example of an embodiment of the present invention;

FIG. 10 is a display of overall hazards generated by an example of an embodiment of the present invention;

FIG. 11 is a display of progress in remedial training to reduce hazardous practices generated by an example of an embodiment of the present invention;

FIG. 12 is a display of a dashboard showing a plurality of users' status generated by an example of an embodiment of the present invention; and

FIG. 13 is a display of spine hazardous movements compared to spine safe movements generated by an example of an embodiment of the present invention.

### DETAILED DESCRIPTION OF EMBODIMENTS OF THE INVENTION

A worker may not be aware that they are exhibiting poor manual handling practices, such as a poor lifting posture, a high frequency of lifts during a work shift, carrying a load for an extended period, not taking a rest after exertions, over exertion and not resting for long enough. For a worker to reduce the risk of injury, continuous monitoring of the body part movements can be conducted and a data set representing the movements can be created and analysed to identify hazardous movements and alerts when hazards are identified. Additionally, the (instantaneous/real-time) data set can be analysed longitudinally (over time) and a resultant report can be made available for the worker and an employment organisation to understand the worker's manual handling practices and potentially to make changes that reduce risk of injury.

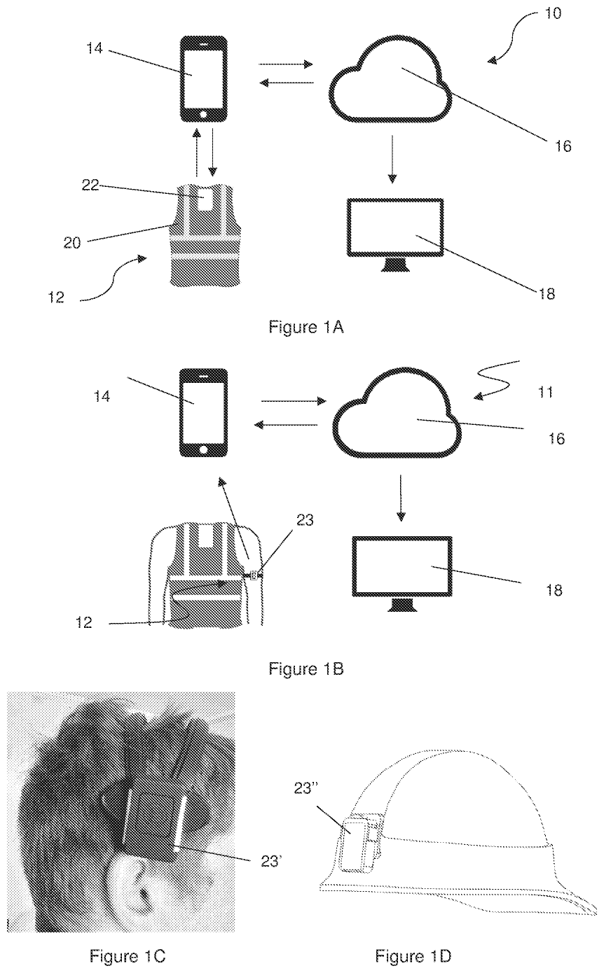

FIG. 1 illustrates a system 10 for monitoring the body movements of a torso of a user. The system 10 comprises a wearable item 12 that is worn by a user and which is able to communicate with a personal computing device 14 that is able to connect to and communicate with a remote computing resource 16, and in an embodiment a remote computing device 18. The wearable item 12 shown in FIG. 1 is a vest, however in an alternative, the wearable item is a clip-on tag that attaches to the user or their clothing. The wearable item 12 comprises a monitoring device 22 for collecting a data set representing body movement of the user over time. The wearable item is for attaching the monitoring device 22 to the user and could take on other forms, such as for example, a strap. Each of the computing devices 14, 18 can be a smartphone, a tablet computer, a laptop computer, a desktop computer or the like. The remote computing resource 16 can be a network based (cloud) computer, a remote computer, or a remote server. The remote computing resource 16 can be implemented using any suitable computing device, preferably one with more processing power than the computing devices 14, 18. FIG. 1A shows an alternative system 11, in which the wearable item 12 is a monitoring on device 23 located on an upper arm of the user by a strap. Other parts of the system 11 are similar to those of system 10. FIG. 1B shows a further alternative, in which the wearable item 12 is a monitoring on device 23′ located on a head of the user by a headband. Other parts of the system are similar to those of system 10. In an embodiment the monitoring device 22, 23, 23′ is capable of being used on any of the back of the torso and/or the upper arm and/or the head.

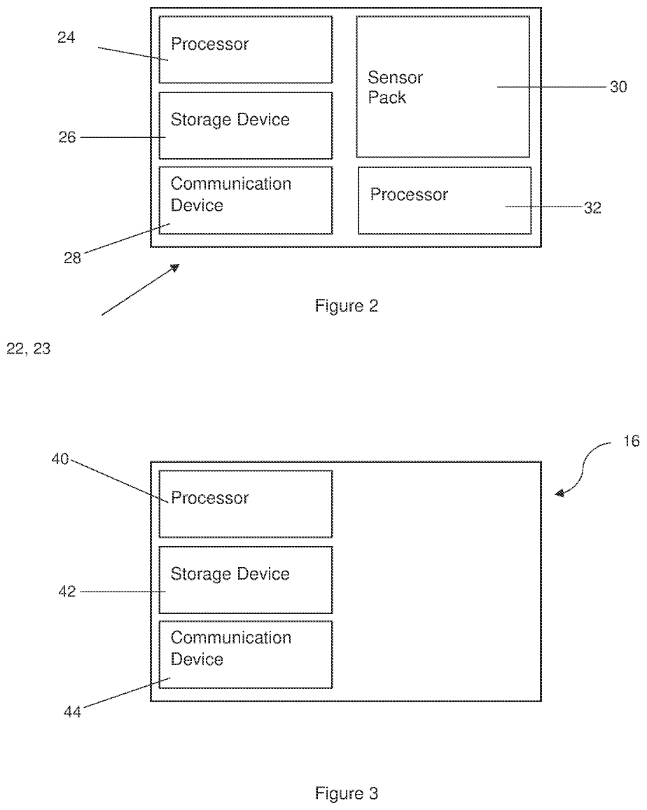

Referring to FIG. 2, the monitoring device 22, 23, 23′ comprises components including a processor 24, a storage device 26, a communication device 28 and a sensor pack 30 comprising one or more sensors. The sensor pack 30 (sensor unit) in this example comprises an inertial sensor and an atmospheric sensor (barometer) 30. Preferably the inertial sensor comprises one or more of an accelerometer and a gyroscope for determining movement of the body part of a user. The processor 24 may comprise a data storage device for storing working data and operating instructions, which may be in the form of non-volatile solid state memory or similar. The storage device 26 comprises a short term storage for storing data from the sensor 30 and/or processed data from the processor 24, and may be in the form of RAM, solid state memory or similar.

The processor 24 may comprise one or more physical, logical or virtual CPUs and is for executing the operating instructions, in the form of a computer program, so as to control the components to operate as the monitoring device 22, as described further below. The instructions may be in the form of firmware, or electronic circuitry, or embedded software, as appropriate and/or convenient. In a preferred form, the components of the monitoring device 22 are low power drawing devices that are powered by a long life battery.

The processor 24 may be configured by operating instructions to operate as one or more of the functional modules described in relation to FIG. 5. The functional modules may also, in addition or instead, and as appropriate and/or convenient, be formed of electronic circuitry. The communication device 28 is typically a wireless network interface, such as a Bluetooth transceiver. Other types of interface are possible, such as IEEE 802.11.

The monitoring device 22 may also comprise a processor 32 for analysing the data set from the sensor 30 to determine when an action or set of actions reaches a safety threshold(s), in which case the user may be alerted. The processor 32 may make a similar analysis to that performed by the remote computing resource 16, as described below, or it may be a simplified version of the analysis. The processor 32 may be a separate processor to processor 24, in a physical, logical, or functional sense, or the processor 24 may be configured to operate as the processor 32.

Referring to FIG. 3, the remote computing resource 16 comprises components including a processor 40, a storage device 42, and a communication device 44. The processor 40 may comprise a data storage device for storing working data and operating instructions, and may be in the form of non-volatile solid state memory or similar. The storage device 42 comprises a long term storage for non-volatile storing of data received via the communication device 44 or processed data from the processor 40. The storage device 42 may be in the form of non-volatile solid state memory, hard disk drive(s) or similar.

The processor 40 may comprise one or more physical, logical or virtual CPUs and is for executing the operating instructions, in the form of a computer program, so as to control the components to operate as the remote computing resource 16, as described below. The instructions may be in the form of firmware, or electronic circuitry, or software, as appropriate and/or convenient.

The processor 40 may be configured by operating instructions to operate as one or more of the functional modules described in relation to FIG. 6. The functional modules may also, in addition or instead, as appropriate and/or convenient, be formed of electronic circuitry. The communication device 44 is a computer network interface.

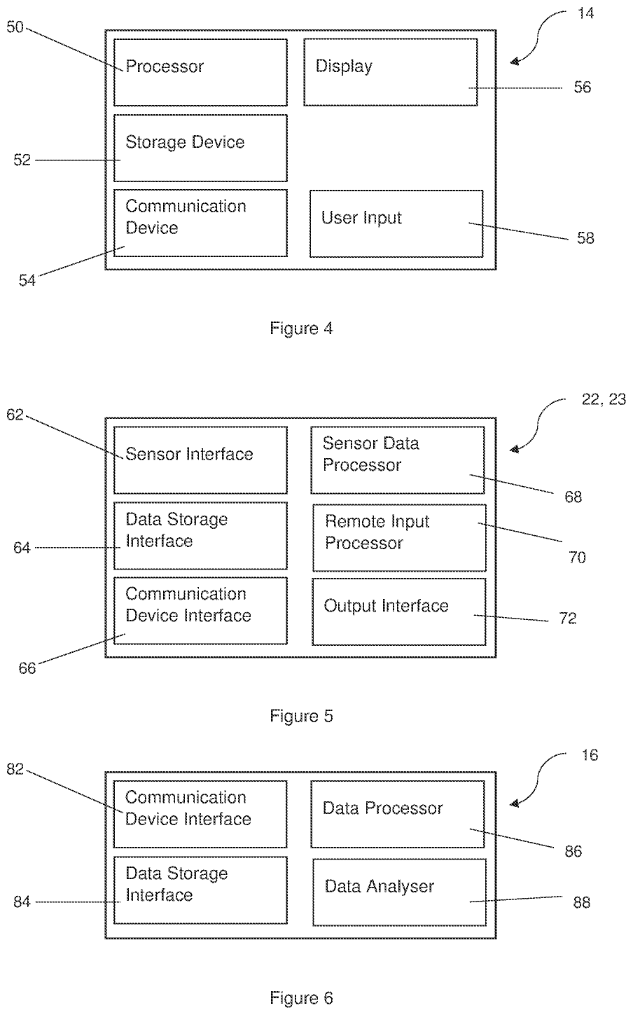

Referring to FIG. 4, the personal computing device 14 comprises components including a processor 50, a storage device 52, a communication device 54, a display 56 and a user input 58, such as a touch screen. The processor 50 may comprise a data storage device for storing working data and operating instructions, and may be in the form of non-volatile solid state memory or similar. The storage device 52 comprises a long term storage for non-volatile storing of data received via the communication device 54 or processed data from the processor 50, and the storage device 42 may be in the form of non-volatile solid state memory or similar.

The processor 50 may comprise one or more physical, logical or virtual CPUs and is for executing the operating instructions, in the form of a computer program, so as to control the components to operate as the personal computing device 14, as described below. The instructions may be in the form of firmware, or electronic circuitry, or embedded and/or installed software, as appropriate and/or convenient.

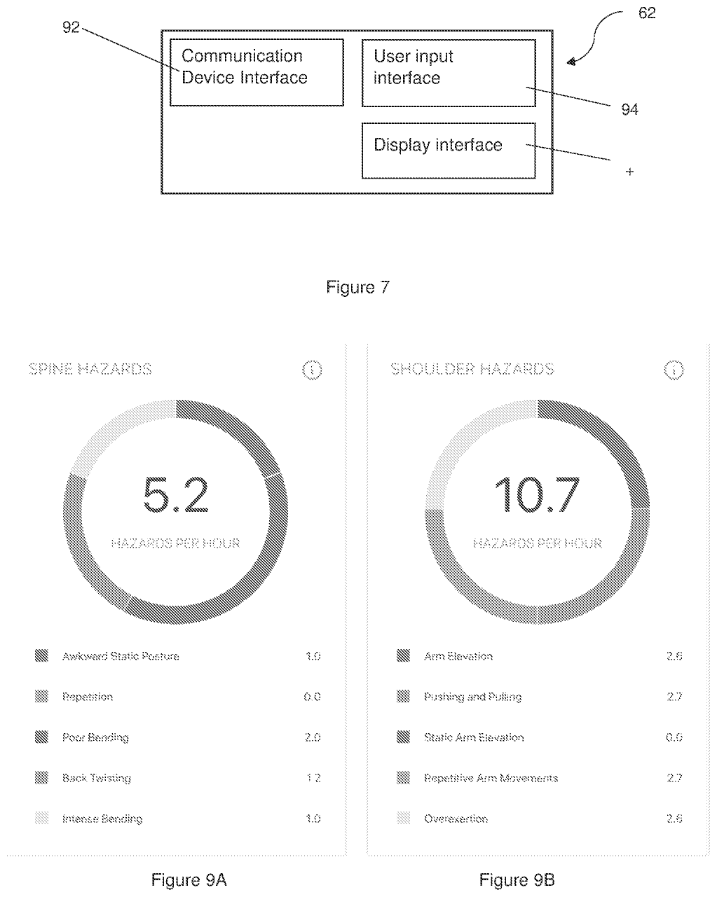

The processor 50 may be configured by operating instructions of an application computer program to operate as one or more of the functional modules described in relation to FIG. 7. The functional modules may also, in addition or instead, as appropriate and/or convenient, be formed of electronic circuitry. The communication device 54 is typically a cellular telephone network interface and/or a Wi-Fi interface and/or a Bluetooth interface.

Referring to FIG. 5, the monitoring device 22, 23, 23′ comprises functional modules including a sensor interface 62, a data storage interface 64, a communication device interface 66, a sensor data processor 68, a remote input processor 70 and an output interface 72. The sensor interface 62 takes a raw sensor input signal from each sensor of the sensor pack 30 and provides it as sensor data to other functional modules, such as the data storage interface module 64 which stores the sensor data in the storage device 26. The sensor data processor 68 is arranged to access sensor data from the sensor pack 30 via the sensor interface 62, and/or to retrieve data from the storage device 26 via the data storage interface 64. The sensor data processor 68 may perform some or all of the processing of the data described below, such as to determine risk and/or characteristics of the movement. None, some or all of the processing may be off loaded to the mobile device 14 or remote computing device 16. Data from the remote computing resource 16 may be received via the communication device interface 66. This data may be stored in the storage device 26 via the data storage interface 64. It may also be processed by the remote input processor 70. Data may be output to the personal computing device 14 (and then in turn to the remote computing resource 16) via an output interface 72.

Referring to FIG. 6, the remote computing resource 16 functional modules include a communication device interface 82, a data storage interface 84, a data processor 86, and a data analyser 88. The communication device interface 82 is arranged to send and receive data to/from the personal computing device 14 or the remote computing device 18. The data storage interface 84 stores the sensor data in the storage device 42. The data processor 86 is arranged to access stored data from data storage device 42 via the data storage interface 84. Typically the processing involves determining the risk factors, as described below. The determined risk factors may then be stored in the data storage device 42 via the data storage interface 84. The data analyser 88 is arranged to access stored data from data storage device 42 via the data storage interface 84, or the risk factors data from the data processor 86 to analyse it. Typically the analysis involves determining whether one or more of the risk factors reach a threshold, as described below. Risk factor data or threshold comparison data may be output to the personal computing device 14 (and then in turn to the monitoring device 22) via a communication device interface 82.

Referring to FIG. 7, the personal computing device 14 comprises functional modules including a communication device interface 92, a user input interface 94, and a display interface 96. The communication device interface 92 is arranged to send and receive data to/from the monitoring device 22 or the remote computing device 18. The user input interface 94 receives inputs from the user. The display interface 96 provides information to the user.

The steps of the method of monitoring the body part movements of a user may be implemented by the monitoring device 22, 23, 23′, personal computing device 14 and remote computing resource 16 operating by, for example, their respective processors 24, 40 and 50 executing respective instructions of a respective computer program so as to operate as described further below.

In an embodiment, the measurement device 22, 23, 23′ collects a data set representing movement over time of a body part of a user. The measurement device 22, 23, 23′ may process the data using its processor 24, 32. The data set or processed data set is transmitted to the personal computing device 14. The personal computing device 14 may further process the data and/or conduct analysis on the data set or processed data set. The personal computing device 14 transmits the data set or processed data set to the remote computing resource 16 which analyses/further analyses the data set/processed data set. The analysis/further analysis determines whether the movement is a hazardous movement. Each hazardous movement is recorded and are collated to produce a pattern of hazardous movements which provide an indication of the level of risk for incurring injury in the movement the user has performing. The indication of the level of risk may be the number of hazardous movements in a given period. The indication of the level of risk is transmitted to the personal computing device 14, which delivers the indication to the user. The personal computing device 14 may transmit the indication of the level of risk to the wearable item 12 for it to deliver the indication to the user. The indication of the level of risk may be made available to the remote computing device 18 in the form of a website, for example.

In a preferred embodiment, the indication is a visual indication. The visual indication may be in the form of a graph indicating the plurality of risk scores plotted over a collection time frame of the data set. In another embodiment, the visual indication is in the form of a written report which may include the visual graph. In a further embodiment, the visual indication is in the form of lights which signify various risk levels. In an embodiment the visual indication may be the number of high risk movements in a given period (eg per hour or per work shift).

In other embodiments of the invention, the indication may be an audio indication or a tactile indication. The audio indication is preferably in the form of an aural report, providing information and alerts to the user. In another embodiment, the audio indication is in the form of a tone that may notify a user of various risk levels based upon volume and repetitions. The tactile indication is preferably in the form of a single vibration or a series of vibrations that may notify a user of various risk levels based upon intensity and repetitions.

In an embodiment of the invention, the remote computing resource 16 is a network based (cloud) computer configured to determine whether a risk score determined from the data sets has exceeded a risk threshold. The risk threshold is a determined value that indicates when the user is participating in a manual handling activity that has the potential to cause serious injury. If the risk threshold has been exceeded, the remote computing resource 16 will generate an alert which is transmitted to the personal computing device 14 and to the wearable item 12. The alert is in the form of a noticeable indication, such as an alarm tone from the speakers of the personal computing device 14 or vibrations from the monitoring device 22 of the wearable item 12.

In another embodiment of the invention, the system 10 comprises a plurality of wearable items 12 which collect a plurality of data sets representing body movements over a period of time from a plurality of users. The plurality of data sets is transmitted to a plurality of personal computing devices 14, which are each connected to the remote computing resource 16. The remote computing resource 16 is configured to receive the plurality of data sets and analyses each data set separately to determine an indication of the level of risk of incurring an injury for each data set. The plurality of indications of risk may be collated to produce a work place assessment of the level of risk for incurring injury. The indicator of level of risk may be the number of high risk movements in a time period. Each indication of the level of risk is transmitted to each personal computing device 14 for delivery to each user. Each personal computing device 14 may transmit the indication to each wearable item for delivery to each respective user.

The remote computing device 18 is configured to receive an aggregated report from the remote computing resource 16. In an embodiment the aggregated report is created by the remote computing resource 16 by collating information gathered by the data sets and the plurality of risk scores.

FIG. 1 shows the wearable item 12 in the form of a vest 20, covering the torso of the user, having a monitoring device 22 or a clip-on tag. FIG. 1A shows the wearable items 12 in the form of a monitoring device 23 strapped to an upper arm by an arm band. The devices 22, 23, 23′ may be configured specifically for the respective body part, or they may be the same device which is able to be informed which body part it is attached to so as to work appropriately for that body part, or it may self-determine the body part it is attached to due to the unique type of movements each body part undertakes.

The monitoring device 22, 23, 23′ may transmit measurement data to the computing device 14 by a direct connection, such as a Bluetooth wireless connection, or other suitable connection, or it may be relayed to the computing device 14 via a receiver device, such as a Bluetooth beacon. Notably the monitoring device 22, 23, 23′ is in a single package. This is notably distinct from the two (or more) monitoring devices at spaced apart locations on the body. The monitoring device 22 may work in combination with another monitoring device (such as 23), which might provide an enhancement to the monitoring described herein by monitoring different body parts, but each monitoring devices does not require another monitoring device for sensing movement etc. of the body part it monitors.

In an embodiment, the sensor pack 30 comprises a multi-axis accelerometer, capable of detecting magnitude and direction of acceleration in order to determine movements of the respective body part of the user that the device 22, 23, 23′ is attached to. The sensor pack 30 also comprises a gyroscope capable of detecting angular velocity of the respective body part of the user that the device 22, 23, 23′ is attached to. Preferably the sensor pack 30 further comprises an atmospheric pressure sensor (high sensitivity barometer) configured to measure changes in altitude of the device 22, 23, 23′. Additional sensors such as magnetometers, or further accelerometers, or gyroscopes may be included in the sensor pack 30. In an embodiment the sensors are sampled by the sensor interface 62 at a frequency determined by the previous measurement, such that when there is a substantial change in the measurement the sample rate is increased and then when the change is slower the sample rate is decreased. This can conserve battery life. In addition, or instead, the accelerometer is sampled by the sensor interface 62 after period of time has lapsed, such as for example 20 ms (producing a sampling rate of 5 Hz). Other sensors can be sampled at a slower rate or can be in a sleep mode when the accelerometer is not experiencing acceleration (other than gravity). In a preferred form the sample rate is about 1.25 Hz in sleep mode and 52 Hz in active mode.

As the sensor pack 30 measures attributes of the movement of the body part, the

processor 24 processes the measurements of body part movements. In an embodiment movement of the body part is determined to be an event and its parameters are determined by processing the measurements form the sensor pack 30. For example, the event might be bending, and the parameters include the maximum angle of bending and the maximum angle of twisting in the event. It may also determine a duration of motion in the event, and one or more durations of stages of movement in the event. These are determined, collected and stored in the storage device as the data set. In an embodiment, periodically the data set can be transmitted, in the form of a signal, to an external source. The process will repeat according to a pre-set time interval. The time interval may be altered according to instructions from the user or from an external source, such as the remote computing resource 16.

The data set comprises values corresponding to each specific body part movement as measured by the sensor pack 30. The primary body part movements of the back are angular movements in a vertical plane, and rotational movements. These movements are used to determine a bending angle and a twisting angle of the user's torso, which provides information regarding trunk flexion/extension and rotation. The bending angle is measured by the change in angle relative to the horizontal of a first reference point, indicating the neutral stance of the user. The twisting angle is measured by the change in angle relative to a line of gravitational force of a second reference point, indicating the neutral stance of the user. The data set further comprises values relating to the static posture time. The static posture time is the time during which substantially no movement of the first and second reference points occurs.

The primary body part movements of the upper arm are angular movements in a vertical plane and a horizontal plane, relative a shoulder socket joint. Additionally, there may also be rotational twisting centred on the longitudinal axis of the upper arm. These movements may be used to determine one or more of: 3D position in space of the upper arm or a portion thereof, yaw and pitch angles, and optionally a twisting (roll-like) angle, of the user's upper arm with respect to the shoulder joint, which provides information regarding the healthy or hazardous use of the user's shoulder.

The primary body part movements of the head are angular movements in a vertical plane and a horizontal plane, as well as twisting, through the neck, relative to around the top of the thoracic portion of the spine. These movements may be used to determine one or more of: 3D position in space of the head thereof, yaw pitch and roll angles through the neck, which provides information regarding the healthy or hazardous use of the user's neck or spine.

The data set further comprises values relating to the static posture time of the upper arm or head. The static posture time is the time during which there is substantially no movement of the upper arm/head.

The monitoring device may be attached to other body parts in which movement of the body part or a related body part is to be monitored for healthy or hazardous movement. For example, the body part may the hand and the related body part may the wrist.

The monitoring device 22, 23, 23′ comprises a transmitter for transmitting the collected data and is configured to transmit the data sets to external sources, such as the personal computing device 14. In the present invention, the monitoring device 22 will automatically transmit the data sets when within range of the personal computer device 14. However, the transmission of the data sets may occur according to specific instructions or prompts. In an example, the monitoring device 22 may transmit the data sets to the personal computing device 14 upon receiving a broadcast from an electronic beacon, such as a Bluetooth low energy beacon. In a further example, the monitoring device 22 may transmit the data sets to the personal computing device 14 upon receiving instructions from the personal computing device 14. In another embodiment, the monitoring device 22 may be configured to transmit the data set to the remote computing resource 16.

In the current invention, the monitoring device 22, 23, 23′ is configured to provide means of indications to the user. The means of indications are used to indicate to the user the indications of the levels of risk determined by the remote computing resource 16, or the alert generated by the remote computing resource 16. The means of indications may be in the form of a speaker and/or a vibration device.

The monitoring device 22, 23, 23′ may be ruggedized to suit the environment in which it will be operating.

The personal computing device 14 is preferably configured to remain in continuous or frequent connection with the monitoring device 22, 23, 23′ so to receive the data sets from the wearable item 12. Alternatively, the monitoring device 22, 23, 23′ will choose when to connect to the personal computing device 12. This may be periodically, or at the conclusion of each or a number of events. In another alternative, the personal computing device 14 will initiate connection to the monitoring device when the user interacts with the device 14, whereupon stored data since the last download will be transmitted to the personal computing device 14. In the present invention, the personal computing device 14 is used as part of a relay network to transmit, in the form of a signal, the data sets from the monitoring device 22, 23, 23′ to the remote computing resource 16 and instructions from the remote computing resource 16 to the monitoring device 22, 23, 23′. The personal computing device 14 is further configured to receive the data sets from the wearable item 12. The data sets and the indications of levels of risk are stored on a storage device of the personal computing device 14 and indications of levels of risk and/or information, such as alerts, may be displayed to the user for reference of their manual handling activities.

In an embodiment of the invention, the personal computing device 14 is further configured to receive the alert generated from the remote computing resource 16 for transmitting to the wearable item 12 and to indicate to the user of their participation in a manual handling activity that has the potential to cause injury. The indication issued by the personal computing device 14 may be in the form of a visual alert, an audio alert, and/or tactile feedback.

In an embodiment of the invention, the personal computing device 14 is further configured to transmit location data and/or other identification data with the data set to the remote computing resource 16.

In the present invention, the remote computing resource 16 is configured to receive and analyse the data set from the wearable item 12 to determine the processed data set and/or analysed data set.

The analysis comprises determining an orientation of the body part; maintaining a record of a state of movement of the body part; determining whether the body part has a changed state of movement and updating the maintained record of the state of movement when the body part has the changed state of movement; classifying the movement of the body part according to the orientation of the body part, the current state of movement of the body part and the data during the current state of movement of the body part.

In an embodiment, determining the orientation of the body part comprises applying one or more filters to the measured movement of the body part. Preferably, the one or more filters comprise a Madgwick filter.

In an embodiment, determining whether the body part has the changed state of movement comprises determining whether the body part has started moving, or has finished moving, and the direction of movement relative to the determined orientation of the body part when the body part is moving.

In an embodiment the analysis comprises determining an angle of an arc traced by movement of an extent of the body part (angle of movement). For example, the extent may be an axial length of the upper arm and the arc may be traced by the elbow when the pivot point of the movement is the shoulder socket. Alternatively, the extent may be the length of the spine and the arc may be traced by the neck when the pivot point is at the waist. In an embodiment the analysis comprises determining an angle of an arc traced by movement of the body part when it changes orientation (angle of orientation). For example, the arm may be in a horizontal orientation and changes to a vertical orientation (such that the angle is 90 degrees). The orientation may be in one or two dimensions. Alternatively, the back may be vertical (shoulder blades facing horizontal) and the change in orientation may be to horizontal, in a straight bend down. Additionally, the orientation of the shoulder blades may be at an angle to vertical when there is also twisting.

The measurements may be taken from a neutral starting position, such as standing straight or sitting straight with movement tracked in 3D space from the neutral starting position.

In an embodiment the analysis comprises determining whether a determined state change is a false positive. In an embodiment determining of the false positive comprises analysing one or more of: a duration of a detected movement, a change in the angle of movement, an orientation of the body part, a rate of change of the angle of movement, a rate of change of the angle of orientation. In an embodiment determining the false positive comprises determining whether there is an atmospheric pressure change. In an embodiment the pressure change is measured to determine whether the body part altered it altitude as determined by the change in atmospheric pressure.

In an embodiment the classification comprises determining a tilting or movement technique performed by the body part. In an embodiment determining the technique comprises analysing the change in the angle of movement, and/or the orientation of the body part and/or determining whether there is an atmospheric pressure change.

In an embodiment the classification comprises determining whether there is a transition from standing to sitting or a transition from sitting to standing. In an embodiment determining whether there is a transition to/from sitting comprises providing the inertial motion data and/or data derived therefrom to a trained classifier and receiving the output of the classifier as the determination. The classifier may be trained using machine learning techniques with data from a plurality of different users or may be trained with data specific to the one user. In an alternative embodiment determining whether there is a transition to/from sitting comprises applying a set of one or more condition checks based on the inertial motion data and/or data derived therefrom.

The data set is comprised of values determined by the bending angle/twisting angle of the back, if applicable, the static posture time, an amount of exertion, a period of exertion, a period of rest, and a period without rest, jerkiness of movement, repetition of a type of movement.

An event is defined as a movement of the body part as determined from the data set. An noted above filtering of the data set (such as by use of a Madgwick filter) ‘cleans up’ the data and false positive event detection can eliminate apparent events which do not meet the criteria for being a true event (at least as determined by the criteria). For example, when lifting the user may bend down and then up. The sensor device will change its position and its relative height. If some of the data indicates bending down, but for example the height does not change, it may be a movement, but not a lift. A ‘false positive’ detection of lift by analysis of some of the data can be rejected because all of the criteria for a lift are not met (eg. no change in height as determined by the external air pressure sensor). The data ‘falsely’ indicating a lift may be measuring movement that merely appear to be a lift but is not.

The amount of exertion is determined by the magnitude of deviance from the first and second reference points. The period of an action is determined by the time frame in which exertion is determined to have occurred. The period of rest is determined by the time period between actions, or if actions are repeated frequently a prolonged period between when the last action occurred and when a new set of actions is commenced. The period without rest is determined by the time period between rests.

The risk of injury is determined by calculating risk factor identifying events that reflect an aspect of the movement. They are determined and then used to produce a risk score representing the level of risk at a current state of time. Over time a plurality of risk scores may be generated. The resultant plurality of risk scores is collated to provide an indication of the level of risk of activities over time. Additionally, each risk score may be compared to a risk threshold value, which will result in the generation of the alert, indicating that the movement event is hazardous. The remote computing resource 16 may be further configured to transmit the plurality of indications of the level of risk and the alerts to the wearable item 12 and the personal computer device 14.

The remote computing resource 16 is further configured to receive a plurality of data sets from a plurality of wearable items 12 to analyse and determine a plurality of risk scores for the user of each wearable item 12. The remote computing resource 16 will subsequently aggregate the plurality of risk scores for each wearable item 12 to produce an aggregated report that is transmitted to the remote computer 18. The remote computer 18 is typically the computer assigned to a safety officer or other workplace official that monitors workplace safety. In the present invention, the aggregated report provides a general indication of the levels of risk associated to the workers from the workplace organisation and/or from specific departments/sections of the workplace organisation. However, the aggregated report may include detailed information of individual users and their activities as required.

In an embodiment of the invention, the aggregated report may include additional information, such as location data and/or identification data to provide relevant information to the general indication of the levels of risk. In an example, the aggregated report may combine location data with the general indication of the levels of risk to determine workplaces with higher occurrences of dangerous activities.

The remote computing resource 16 is controlled by a computer program executable by a computer of the remote computing resource 16 embodied on a computer readable media. The computer program comprises instructions to configure the remote computing resource 16 as a special purpose machine that performs the functions previously described.

The personal computer device 14 is intended to be arranged as part of the system 10 which includes the monitoring device 22, 23, 23′ and the remote computing resource 16. However, in an embodiment of the invention, the system may be comprised only of the monitoring device 22, 23, 23′ and the personal computer device 14. In this embodiment, the personal computer device 14 is configured to perform the functions of the remote computing resource 16 in addition to its own functions. Indeed the monitoring device 22, 23, 23′ and/or computing device 14 can be configured to provide an alert of a hazardous movement near instantaneously (commonly referred to as ‘in real-time’).

The monitoring device 22, 23, 23′ is intended to be arranged as part of the system 10, which includes the personal computer device 14 and the remote computing resource 16. However, in an embodiment of the invention, the monitoring device 22, 23, 23′ may be configured to perform part of the functions of the remote computing resource 16 in order to consistently generate the alert of dangerous manual handling activities.

In an embodiment of the invention, the monitoring device 22, 23, 23′ may be configured to perform the functions of the remote computing resource 16.

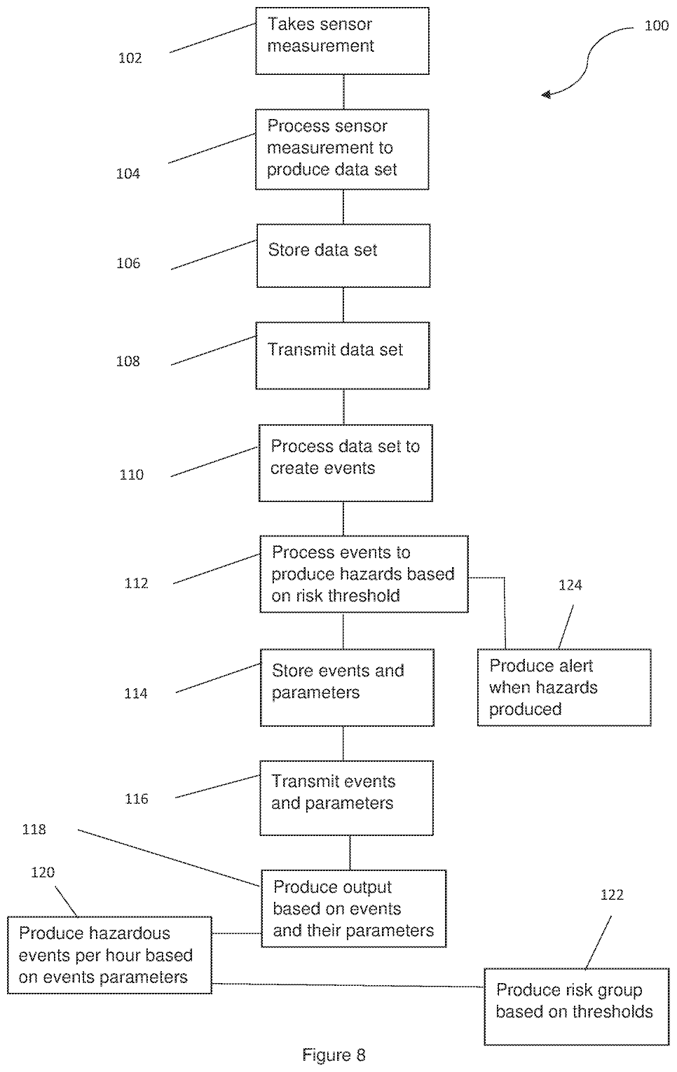

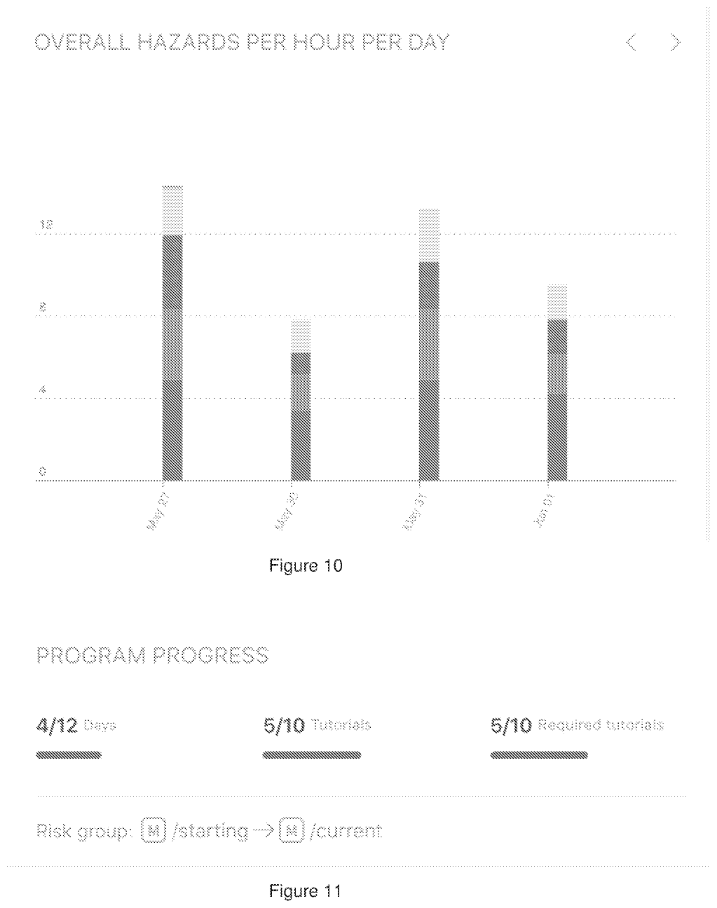

A method of operation 100 and use of the system 10 used to monitor the core body movements of the user will now be described in more detail with reference to FIG. 8.

The user wears the monitoring device 22, 23, 23′ on a body part to be monitored and conducts the typical manual handling actions required for their job. The monitoring device 22 takes measurements 102 using the sensor pack 30. The raw measurements may be processed 104 into a consistent format for a data set representing the body part movements as measured by the monitoring device 22 over time. The data set is stored 106 and processed prior to being transmitted 108 to the personal computer device 14 of the user. The data set is stored on the personal computer device 14 prior to transmission to the remote computing resource 16.

The data set is processed 110 by the monitoring device 22, 23, 23′ or the remote computing resource 16 to determine risk of injury of the body part and/or to track the movement of the body part. In particular the data set is processed to determine an event has occurred. The event may be characterised by calculating parameters from the data set. For example, the event of a lift may be determined and the amount of excursion required for the lift, its duration and other characteristics about the lift may be determined. In some embodiments this may include calculating 112 whether a risk threshold is reached/exceeded. When the threshold is exceeded a hazard alert is generated 120. For example, when too much excursion is used, or when the lift is both bending and twisting to a risky extent.

The generation of the alert informs the user that they are engaging in manual handling activity that may potentially lead to injury. Alternatively, when there is a number of high risk movements a given period of time, this may be compared to a threshold or otherwise determined that there has been too many risky movements over time, then an alert may be generated. The alert warns the user of their dangerous activity using indications such as visual, aural and/or tactile notifications.

The generated events and calculated parameters are stored 114 and transmitted to the mobile device 14 and preferably then on to the remote computing resource 16. The mobile device 14 and/or the remote computing resource 16 generate 118 an output based on events and their parameters, the number of hazardous events per hour based on event parameters is produced 120 and risk groups are determined 122 based on thresholds. These determinations are produced as an output report to the user, such as shown in FIGS. 9A 9B, 10, or 13. The report is subsequently transmitted to the remote computer 18 of the safety officer or other official for review and the worker's individual report may be transmitted to their person computing device 14 for inspection.

In an embodiment accelerometer measurement are modelled according to the determined body part the sensor is attached to. Abstract features may also be used in some embodiments to assess the risk of a movement or series of movements, such as:
max (v), min (v), mean (v), median (v), where v is substituted by each component of accelerometer data (aX, aY, aZ) and accelerometer magnitude, where

$$accelerometer magnitude = \sqrt{{aX}^{2} + {aY}^{2} + aZ \hat{} 2} - 1 ;$$

and

The resulting feature list is:
1. Time of the body bending down
2. Time of the body rising up
3. Time of the body in near static posture
4. max(v), where v is bending angle
5. min(v), where v is bending angle
6. mean(v), where v is bending angle
7. median(v), where v is bending angle
8. max(v), where v is twisting angle
9. min(v), where v is twisting angle
10. mean(v), where v is twisting angle
11. median(v), where v is twisting angle
12. max(v), where v is aX
13. min(v), where v is aX
14. mean(v), where v is aX
15. median(v), where v is aX
16. max(v), where v is aY
17. min(v), where v is aY
18. mean(v), where v is aY
19. median(v), where v is aY
20. max(v), where v is aZ
21. min(v), where v is aZ
22. mean(v), where v is aZ
23. median(v), where v is aZ
24. max(v), where v is accelerometer magnitude
25. min(v), where v is accelerometer magnitude
26. mean(v), where v is accelerometer magnitude
27. median(v), where v is accelerometer magnitude
28.-48. 20 coefficients of FFT over accelerometer magnitude.

A model for predicting the relative object weight is determined from the signal features, as input data, and the resulting features, as output data intended to be produced by the model. In an embodiment the model is a decision tree. A regression model is used to determine the characteristics of the decision tree from the input data and the output data. In an embodiment the regression model uses a gradient boosting machine to realize the decision tree. The model may be refined over time.

A training data set with target values of relative weight (mass of an object divided by human mass) of an object which have been picked up or placed to the ground is used by the regression model to determine/refine the characteristics of the model.

Constants were defined with respect to the training set. The algorithm is validated and optimized by using cross-validation techniques.

In an embodiment the amount of force used during exertion is determined by measuring the duration at which there is bending. In particular the rate of change is measured. The rate of change may be a first order derivative (speed), second order derivative (acceleration) or third order derivative (jerk) measurement. The theory behind this is that when straining, that is applying a lot of force to an object, movement is minimal or slow until momentum in the change of position (angle) is achieved. For example, a change in acceleration can be used to infer the amount of force exerted and thus the weight or load being moved in an action.

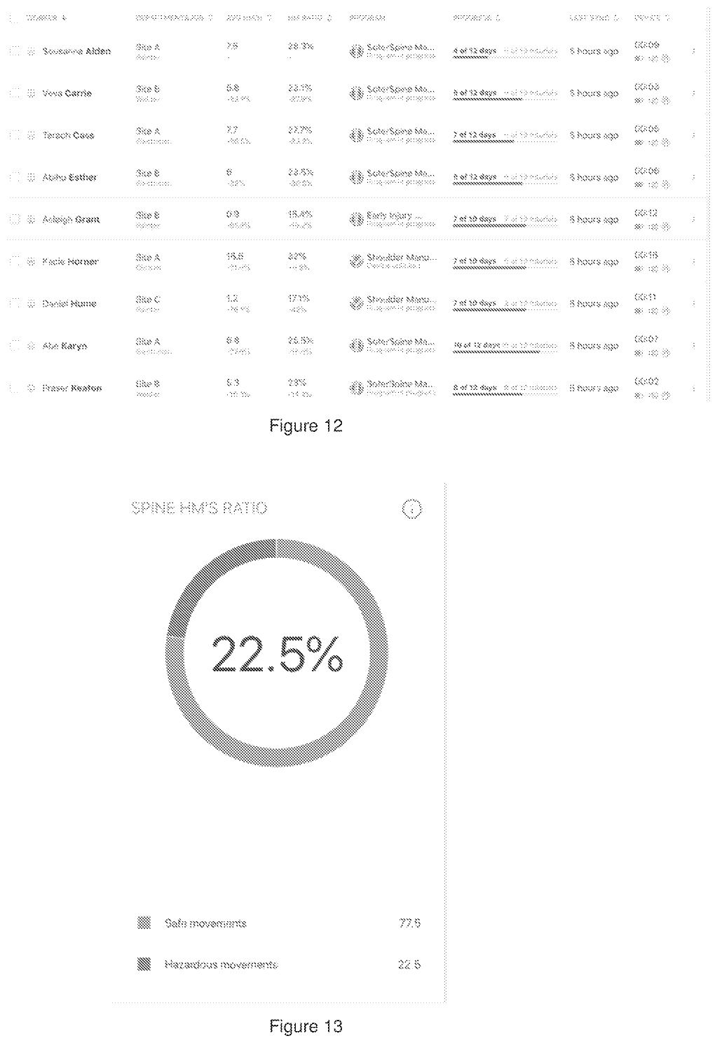

The reports provide the advantage of informing the safety officer or other official and the worker of the level of workplace safety and the adoption of risk control mechanisms. The report may also include recommendations based upon the aggregated information. An example organisation level dashboard report such as shown in FIG. 12 can present an overview of hazardous activities and progress in hazardous activity reduction over time.

The report and/or thresholds may be adjusted to each individual user, by factoring in user specific parameters, such as exposure to vibration, side bending, force (Distance between the load and the body, weight of the load, speed of bending and coming up), fatigue, age, weight, height, BMI, and medical history.

In an alternative each movement is determined to be low or high risk or a series of movements have be determined to comprise a number of high risk movement in a period of time, for example per hour or per work period (shift). This high risk movement or the number of high risk movements over time can be communicated to the person working and/or in a safety report in a workforce.

Events are determined according to body part, such as arm or back or head. Accordingly the device 22, 23, 23′ is chosen according to the body part it is to be attached to, or the device 22, 23, 23′ is told which body part it is attached to (eg by entering a setting) or the device is calibrated and is able determine by monitoring the movement during calibration which body part it is attached to. The latter is typically conducted by feeding the data set into a trained machine learning model which outputs the body part it is attached to.

The events are then determined when the body part moves. Typically, start of a movement is determined. Then an orientation of the body part at the start of a movement is determined. A record of a state of movement of the body part is maintained and updated.

This includes determining when a movement has paused or has finished. The direction of movement and angle of movement in 3D space may be determined form the data set. The movement of the body part is classified according to the orientation of the body part, the current state of movement of the body part and the data during the current state of movement of the body part. The orientation of the body part may be determined using a filter, such as a Madgwick filter.

The type of movement the body part is making is determined from tracking of the movement of the body part. Characteristics of movement of the body part for each type of movement (movement technique, such as lifting technique) are learnt from the tracking of the movement of the body part. Preferably the characteristics determine a “correct” type of implementation, such as those that are good lifting techniques (bending with your legs instead of lifting with your back), and an “incorrect” type of implementation of the determined type of movement the body part, such as those that may cause injury (lifting with your back instead of bending your legs). Another example is correct sitting posture versus incorrect sitting posture.

When the movement of the body part is undertaken the “correct” type of implementation of the determined type of movement (such as you have lifted by bending your legs) and when the movement of the body part is undertaking the “incorrect” type of implementation of the determined type of movement the body part is making (such as you have lifted with your back, not your legs) according to the tracking of the movement of the body part during each movement. In other words, the actual movement is compared to the characteristics of ‘correct’ and ‘incorrect’ movements.

An angle of an arc traced by movement of an extent of the body part (angle of

movement) may be determined. For example, the extent may be an axial length of the upper arm and the arc may be traced by the elbow when the pivot point of the movement is the shoulder socket. Alternatively, the extent may be the length of the spine and the arc may be traced by the neck when the pivot point is at the waist.

An angle of an arc traced by movement of the body part when it changes orientation (angle of orientation) may be determined. For example, the arm may be in a horizontal orientation and changes to a vertical orientation (such that the angle is 90 degrees). The orientation may be in one or two dimensions. Alternatively, the back may be vertical (shoulder blades facing horizontal) and the change in orientation may be to horizontal, in a straight bend down. Additionally, the orientation of the shoulder blades may be at an angle to vertical when there is also twisting.

A neutral starting position of a movement of the body part may be determined. Determination of the angle(s) may be taken from the neutral starting position, such as standing straight or sitting straight, with movement tracked in 3D space from the neutral starting position.

False-positive movements of the body part may be determined using the collected data. In particular, determining of the false positive comprises analysing one or more of: a duration of a detected movement, a change in the angle of movement, an orientation of the body part, a rate of change of the angle of movement, a rate of change of the angle of orientation. In an embodiment, determining the false positive comprises determining whether there is an atmospheric pressure change over the course of the movement of the body part which is indicative of whether the body part altered its altitude as determined by the change in atmospheric pressure.

A tilting or movement technique performed by the body part may be determined by analysing the change in the angle of movement, and/or the orientation of the body part and/or determining whether there is an atmospheric pressure change.

Classification of the movement may be determined when there is a transition from standing to sitting or a transition from sitting to standing. Determining whether there is a transition to/from sitting comprises providing the inertial motion data and/or data derived therefrom to a trained classifier and receiving the output of the classifier as the determination or a precursor thereof. In an alternative embodiment, determining whether there is a transition to/from sitting comprises applying a set of one or more condition checks based on the inertial motion data and/or data derived therefrom.

A state of the body part may be determined, where for example, the state is one or more of: moving, lifting, bending, twisting, twisting while bending and stationary.

The type of movement may be determined to be jerky, or not.

Repetition of movement of the same body part movement over a period of time may be determined, such as the number of movements of the body part over a time period (eg. an hour).

Staying in a static position during a movement of the body part may be determined.

A RULA, NIOSH, and/or WISHA assessment may be determined.

A risk of injury by continued performance of the movement of the body part may be determined from the determined state, type of movement and repetition of movement. The risk may be a risk of back injury, a risk of shoulder injury or a risk of back and shoulder injury.

A work pattern may be determined from a plurality of movements by determining the number and amount of intensities of movement. In an embodiment determining a work pattern comprises determining an average amount of time spent moving in each movement and an average of the amount of time spent recovering from each movement.

The following may be determined a shoulder limb position angle, velocity of movement, jerkiness of movement, hazards per hour, frequency of arm elevation, proportion of time arm elevated, intensity of force of movement over time, amount of time spent moving, amount of time spent recovering.

The following may be determined a back position, velocity of movement, jerkiness of movement, twisting while bending angle, bending angle, hazards per hour, frequency of bending, twisting, static posture and intensity movement, proportion of time bending twisting, intensity of force of movement twisting while bending.

Movements may be classified as one or more of the following: arm elevated more than 90 degrees; arm elevated more than 90 degrees for a period in excess of 30 seconds; arm elevated more than 90 degrees more than 2 times a minute; arm elevated for than 90degrees for more than 20% of working time; hazardous pulling and pushing.

Movements may be classified as one or more of the following: twisting of back more than 30 degrees while bending more than 50 degrees; bending more than 90 degrees; bending more than 60 degrees for at least 20 seconds; at least 2 hazardous movement in 2 minutes; jerky movement.

The assorted determination may then be used to determine an immediate or imminent risk of injury, or a long term risk of injury.

Recommended remedial action can be determined based on the determined risk factors and risk determinations. For example, the following recommendation could be made to mitigate against the determined risk factors:
1. Rotation component can be eliminated or reduced. This will reduce the total risk of low back pain;
2. Reduce duration of being in static posture during a load bearing movement;
3. Reduction of the angle of bending;
4. Take a 30 second rest break in the middle of the flexion;
5. Reduction of the frequency of lifting.

FIG. 11 shows progress on remedial activities and training to reduce the number of hazardous movements.

Reporting on movement risks over an extended period of time, rather than or in addition to on individual movements can create postural awareness of the workers. Reporting might be used to suggest kinesiotaping or biomechanic education of the workers.

Modifications may be made to the present invention within the context of that described and shown in the drawings. Such modifications are intended to form part of the invention described in this specification.

1. A system for monitoring body movement, comprising:
an inertial motion sensing unit arranged to measure movement of a body part, the sensing unit arranged to iteratively collect data representing movement of the body part over time;
a processor for:
determining an orientation of the body part based on the data;
tracking movement of the body part based on the data;
maintaining a record of a state of movement of the body part;
determining whether the body part has changed the state of movement and updating the maintained record of the state of movement when the body part has changed the state of movement;
classifying the movement of the body part according to the orientation of the body part, the current state of movement of the body part and the data during the tracked movement of the body part, wherein the classified movement comprises whether the body is: sitting, or standing, or is transitioning between sitting and standing, or transitioning between standing and sitting;
analysing the classified movement and the data to determine risk of injury of the body part.

2. A system according to claim 1, wherein, the processor is configured to determine the type of body part and a type of movement the body part is making from data collected during tracking of the movement of the body part and the body part type.

3. A system according to claim 2, wherein the determined type of movement is classified as being implemented in either a ‘correct’ or an ‘incorrect’ manner.

4-5. (canceled)

6. A system according to claim 1, wherein determining whether the body part has the changed state of movement comprises determining whether the body part has started moving, or has finished moving, and the direction of movement relative to the determined orientation of the body part.

7-8. (canceled)

9. A system according to claim 1, wherein the analysing comprises determining whether a determined state change is a false positive, wherein determining of the false positive comprises analysing one or more of: a duration of a detected movement, a change in the angle of movement, an orientation of the body part, a rate of change of the angle of movement, a rate of change of the angle of orientation.

10-11. (canceled)

12. A system according to claim 9, wherein classifying comprises determining a selected one of a plurality of predetermined tilting or movement techniques performed by the body part.

13. A system according to claim 12, wherein determining the technique comprises analysing the change in the angle of movement, and/or change in the orientation of the body part and/or determining whether there is an atmospheric pressure change.

14. A system according to claim 1, wherein the processor is configured to analyse the data to determine when a lift is occurring and to determine a lifting technique used when a lift is occurring, and the processor is configured to analyse the data to determine arm movement when a lift is occurring.

15. A system according to claim 1, wherein the data is obtained from a single inertial motion sensing unit.

16. A system according to claim 15, wherein the sensing unit is only located on the arm of the user.

17. A system according to claim 15, wherein the sensing unit is only located on the back of the user.

18. A system according to claim 15, wherein the sensing unit is only located on the head of the user.

19. A system according to claim 16, wherein the processor is configured to analyse the data to determine whether a user is sitting, and to determine arm movement when the user is sitting.

20. A system according to claim 1, wherein the processor is configured to determine an intensity of force used in a movement of the body part.

21. A system according to claim 1, wherein the processor is configured to determine a state of the body part, wherein the state is one or more of: moving, lifting, bending, twisting, twisting while bending and stationary.

22. A system according to claim 1, wherein the processor is configured to determine that during movement of the body part, the movement is jerky.

23. A system according to claim 1, wherein the processor is configured to determine a work pattern from a plurality of movements.

24. A system according to claim 23, wherein determining the work pattern comprises determining the number and amount of intensities of movement.

25. A system according to claim 23, wherein determining the work pattern comprises determining an average amount of time spent moving in each movement and an average of the amount of time spent recovering from each movement.

26. A system according to claim 1, wherein the processor is configured to determine a shoulder limb position angle, velocity of movement, jerkiness of movement, hazards per hour, frequency of arm elevation, proportion of time arm elevated, intensity of force of movement over time, amount of time spent moving, and amount of time spent recovering.

27. A system according to claim 1, wherein the processor is configured to determine a back position, velocity of movement, jerkiness of movement, twisting while bending angle, bending angle, hazards per hour, frequency of bending, twisting, static posture and intensity movement, proportion of time bending twisting, and intensity of force of movement twisting while bending.

28. A system according to claim 1, wherein the processor is configured to classify movements as one or more of the following: arm elevated more than 90 degrees;
arm elevated more than 90 degrees for a period in excess of 30 seconds; arm elevated more than 90 degrees more than 2 times a minute; arm elevated for than 90 degrees for more than 20% of working time; and hazardous pulling and pushing.

29. A system according to claim 1, wherein the processor is configured to classify movements as one or more of the following: twisting of back more than 30 degrees while bending more than 50 degrees; bending more than 90 degrees; bending more than 60 degrees for at least 20 seconds; at least 2 hazardous movement in 2 minutes; and jerky movement.

30. A system according to claim 1, wherein the processor is configured to determine a risk of continued performance of the movement of the body part.

31. (canceled)

32. A system for monitoring body movement, comprising:
an inertial motion sensing unit arranged to measure movement of a body part, the sensing unit arranged to iteratively collect data representing movement of the body part over time;
a processor receiving and analysing the data to determine which body part the inertial motion sensing unit is attached to and the risk of injury of the body part and/or to track the movement of the body part.

33-35. (canceled)

36. A system for monitoring body movement, comprising:
an inertial motion sensing unit arranged to measure movement of a body part, the sensing unit arranged to iteratively collect data representing movement of the body part over time;
a processor receiving and analysing the data to determine a work pattern and to determine the risk of injury of the body part and/or to track the movement of the body part, wherein the work pattern comprises counting risks in a movement.

37-40. (canceled)

41. A system for monitoring body movement, comprising:
an inertial motion unit sensor arranged to measure movement of a body part, the sensor arranged to iteratively collect data representing movement of the body part over time;
a processor for receiving and analysing the data, the processor configured to:
determine an orientation of the body part;
maintain a record of a state of movement of the body part;
determine whether the body part has a changed state of movement and to update the maintained record of the state of movement when the body part has the changed state of movement;
classify the movement of the body part according to the orientation of the body part, the current state of movement of the body part and the data during the current state of movement of the body part.

42-43. (canceled)

44. A method of monitoring body movement, comprising:
measuring movement of a body part by iteratively collect data representing movement of the body part over time using a sensor unit;
determining which body part the sensor unit is attached to based on the collected data;
analysing the data to determine risk of injury of the body part or to track the movement of the body part.

45-54. (canceled)

55. A sensor device for monitoring body part movements, comprising:
a housing having an attachment device for attaching the housing to a head of a user only;
an accelerometer mounted in the housing;
a gyroscope mounted in the housing;
an atmospheric pressure sensor mounted in the housing and arranged to measure external atmospheric pressure;
a processor for collecting data of measurements from the accelerometer, and determining movement of body parts of the user including the head and back of the user.

56-57. (canceled)
